(19) United States
(12) Patent Application Publication
(10) Pub. No.: US 2020/0323753 A1
(43) Pub. Date: Oct. 15, 2020

(54) METHOD FOR FORMING A NATURAL HAIR CONDITIONING COMPOSITION

(71) Applicant: ELC MANAGEMENT LLC, Melville, NY (US)

(72) Inventors: Mollie Suzanne HARKNESS, Bloomington, MN (US); John Michael Bohen, III, Cannon Falls, MN (US); Samantha Mae Guertin, Wyoming, MN (US); Camille Tryntje Sasik, Minnetonka, MN (US)

(21) Appl. No.: 16/846,383

(22) Filed: Apr. 12, 2020

Related U.S. Application Data
(60) Provisional application No. 62/833,452, filed on Apr. 12, 2019.

(51) Int. Cl. A61K 8/31; A61K 8/92; A61K 8/73; A61K 8/362; A61K 8/36; A61Q 5/12

(52) U.S. Cl. CPC A61K 8/31; A61K 8/922; A61K 8/731; A61K 8/737; A61Q 5/12; A61K 8/927; A61K 8/362; A61K 8/361; A61K 8/925

(57) ABSTRACT

The present invention relates to a method for forming a silicone-free hair conditioning composition comprising the steps of, first, forming a first phase, the first phase comprising an olive squalane, one or more esters, and one or more waxes. Then, a second phase is formed, the second phase comprising one or more non-silicone conditioning agents, one or more cationic polymers, and water. Next, the first and second phases are combined to form a mixture. And finally, preservatives and minor ingredients may be added to the mixture.

### FIELD OF THE INVENTION

The present invention relates to a method for forming a silicone-free hair conditioning composition. More specifically, it relates to method for forming a hair conditioning composition comprising an olive squalene component which, in combination with the other essential components described herein, imparts exceptional hair conditioning benefits.

### BACKGROUND OF THE INVENTION

Hair shine and/or gloss are attributes that are desired in many hair care products, preparations and compositions. Conventional hair shine/gloss products are made with silicones such as dimethicone, cyclomethicone, phenyltrimethicone and dimethicone copolyol. While these silicones offer very good shine due to their high refractive index, they also offer a heavy conditioning coating to the hair, which will interfere with the effect of styling products on the hair.

Traditional silicone-based shine products, depending on the molecular weight of the silicone used, either evaporate shortly after application, such as when cyclomethicone is used, or leave a heavy oily residue on the hair, such as when dimethicone is used. In these cases, the shine provided to the hair is either transient or may look and feel greasy.

Silicones are known to be used as plasticizers in hair stylers to prevent brittleness of the film, yet any modification of a styler with silicones tends to weaken the holding power of the styler. Silicones are heavy, and their presence in hair care compositions weigh the hair down. Also, the use of volatile silicones results in hair care compositions that provide a fleeting shine, and a tendency to soften hair after application, resulting in a style that does not last long. Moreover, silicone-based hair compositions tend to be costly.

### SUMMARY OF THE INVENTION

The present invention relates to a method for forming a hair conditioning composition which may be free of silicone oils comprising the steps of, first, forming a first phase, the first phase comprising an olive squalane, one or more esters, and one or more waxes. Then, a second phase is formed, the second phase comprising one or more non-silicone conditioning agents, one or more cationic polymers, and water. Next, the first and second phases are combined to form a mixture. And finally, preservatives and minor ingredients may be added to the mixture.

### DETAILED DESCRIPTION OF THE INVENTION

While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level, and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “polymer” as used herein shall include materials whether made by polymerization of one type of monomer or made by two (i.e., copolymers) or more types of monomers.

The term “solid particle” as used herein means a particle that is not a liquid or a gas.

The term “water-soluble” as used herein, means that the polymer is soluble in water in the present composition. In general, the polymer should be soluble at 25° C. at a concentration of at least 0.1% by weight of the water solvent, preferably at least 1%, more preferably at least 5%, most preferably at least 15%.

The term “water-insoluble” as used herein, means that a compound is not soluble in water in the present composition. Thus, the compound is not miscible with water.

The personal care compositions of the present invention comprise a cationically modified starch polymer, an anionic surfactant system, and a cosmetically acceptable carrier. Each of these essential components, as well as preferred or optional components, is described in detail hereinafter.

### Olive Squalane

The compositions herein comprise an olive squalene component. Squalane is a well-known cosmetic ingredient. For example, U.S. Patent Publication No. 2008-0274068 discloses squalene among other oily substances which may be used to improve hair manageability after drying. However, it has now been discovered that particular squalanes behave differently from not only other oily cosmetic ingredients, but also from squalanes derived from different natural sources. For example, squalene may be derived from sugar cane, which is true for squalene sold under the tradename Neossance®. Squalane may also be derived from animal origin, as is described by Korean Patent No. KR101417029. In contrast, olive squalene is derived from olive oil and exhibits distinct characteristics which are essential to the benefits of the compositions herein. In one embodiment, the olive squalane is present at from about 0.01 wt. % to about 20 wt. %, more preferably from about 0.03 wt. % to about 10 wt. %, and most preferably from about 0.05 wt. % to about 2 wt. % of the composition.

### Wax

The compositions herein comprise one or more wax components. Nonlimiting examples of suitable waxes include, for example, microcrystalline wax, paraffin wax, microcrystalline wax, ozokerite, polyethylene wax, ceresin wax, bees wax, candelilla wax, carnauba wax, shellac wax, lanolin wax, montan wax, orange wax, lemon wax, laurel wax and olive wax, beeswax, and mixtures thereof. Particularly preferred waxes include candelilla wax, carnauba wax, and natural or synthetic beeswax. In one embodiment, the wax is present at from about 0.01 wt. % to about 20 wt. %, more preferably from about 0.03 wt. % to about 10 wt. %, and most preferably from about 0.05 wt. % to about 2 wt. % of the composition. Further, it is preferred that e wax component has a melting point of from about 68.5-72.5° C.

### Ester Emollient

The compositions comprise one or more esters which serve as emollients. The ester enhances the feel of the composition when used to treat human hair. Preferably, the ester is a natural ester selected from methylheptyl isostearate, diisostearyl dimer dilinoleate, diisostearyl malate, tricaprylyl citrate, isostearyl isostearate, polyglyceryl-2 triisostearate, polyglyceryl-2 diisostearate, polyglyceryl-2 isostearate, glyceryl ricinoleate, isostearyl palmitate, caprylic/capric triglyceride, mango butter dimer dilinoleyl esters/dimer dilinoleate copolymer, myristyl myristate, oleyl erucate, polyglyceryl-3 diisostearate, triisostearyl citrate, isostearyl stearoyl stearate, Mangifera indica (mango) seed butter, tricaprylin, glucose glutamate, and mixtures thereof. Each of the foregoing are commercially available under various tradenames from, for example, Alzo International Inc.

The ester may be present at a level of from about at from about 0.01 wt. % to about 20 wt. %, more preferably from about 0.03 wt. % to about 10 wt. %, and most preferably from about 0.05 wt. % to about 2 wt. % of the composition.

Particularly preferred is tricaprylyl citrate, available under the tradename Bernel Ester™ TCC from Alzo International Inc.

Fatty Esters

Other suitable organic conditioning oils for use as the conditioning agent in the compositions of the present invention include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are those known as polyhydric alcohol esters. Such polyhydric alcohol esters include alkylene glycol esters.

Still other fatty esters suitable for use in the compositions of the present invention are glycerides, including, but not limited to, mono-, di-, and tri-glycerides, preferably di- and tri-glycerides, more preferably triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Fluorinated Conditioning Compounds

Fluorinated compounds suitable for delivering conditioning to hair or skin as organic conditioning oils include perfluoropolyethers, perfluorinated olefins, and fluorine-based specialty polymers that may be in a fluid or elastomer form. Specific non-limiting examples of suitable fluorinated compounds include the Fomblin product line from Ausimont which includes HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

Fatty Alcohols

Other suitable organic conditioning oils for use in the personal care compositions of the present invention include, but are not limited to, fatty alcohols having at least about 10 carbon atoms, more preferably about 10 to about 22 carbon atoms, most preferably about 12 to about 16 carbon atoms. Also suitable for use in the personal care compositions of the present inventions are alkoxylated fatty alcohols which conform to the general formula:

CH₃(CH₂)ₙCH₂(OCH₂CH₂)ₚOH

wherein n is a positive integer having a value from about 8 to about 20, preferably about 10 to about 14, and p is a positive integer having a value from about 1 to about 30, preferably from about 2 to about 23.

Alkyl Glucosides and Alkyl Glucoside Derivatives

Suitable organic conditioning oils for use in the personal care compositions of the present invention include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

Other Conditioning Agents

Quaternary Ammonium Compounds

Suitable quaternary ammonium compounds for use as conditioning agents in the personal care compositions of the present invention include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in a composition of the present invention include, but are not limited to, Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

Cosmetically Acceptable Carrier

The personal care compositions of the present invention comprise a cosmetically acceptable carrier. The level and species of the carrier are selected according to the compatibility with other components desired characteristic of the product. Generally, the cosmetically acceptable carrier is present in an amount from about 20% to about 95% by weight of the composition. A cosmetically acceptable carrier may be selected such that the composition of the present invention may be in the form of, for example, a pourable liquid, a gel, a paste, a dried powder, or a dried film.

Cosmetically acceptable media useful in the present invention include water and water solutions of lower alkyl alcohols. Lower alkyl alcohols useful herein are monohydric alcohols having 1 to 6 carbons, and preferably are selected from ethanol and isopropanol.

The pH of the present composition, measured neat, is preferably from about 3 to about 9, more preferably from about 4 to about 8. Buffers and other pH-adjusting agents can be included to achieve the desirable pH.

Cellulose or Guar Cationic Deposition Polymers

The compositions may also include cellulose or guar cationic deposition polymers. Cellulose or glactomannan cationic deposition polymers are preferred. Generally, such cellulose or guar cationic deposition polymers may be present at a concentration from about 0.05% to about 5%, by weight of the composition. Suitable cellulose or guar cationic deposition polymers have a molecular weight of greater than about 5,000. Preferably, the cellulose or guar cationic deposition polymers have a molecular weight of greater than about 200,000. Additionally, such cellulose or guar deposition polymers have a charge density from about 0.15 meq/g to about 4.0 meq/g at the pH of intended use of the composition, which pH will generally range from about pH 3 to about pH 9, preferably between about pH 4 and about pH 8. The pH of the compositions of the present invention are measured neat.

Suitable cellulose or guar cationic polymers include those which conform to the following formula:

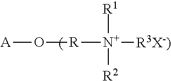

wherein A is an anhydroglucose residual group, such as a cellulose anhydroglucose residual; R is an alkylene oxyalkylene, polyoxyalkylene, or hydroxyalkylene group, or combination thereof; R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose or guar cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA).

Synthetic Cationic Deposition Polymers

While it is preferred that the present compositions are free of synthetic ingredients, it is feasible to include certain synthetic components without drawbacks to performance. When referenced, synthetic ingredients may be included, but it should be appreciated that they are not essential to deliver the cosmetic benefits disclosed herein.

For example, in one embodiment, the compositions may include synthetic cationic deposition polymers. Generally, such synthetic cationic deposition polymers may be present at a concentration from about 0.025% to about 5%, by weight of the composition. Such synthetic cationic deposition polymers have a molecular weight from about 1,000 to about 5,000,000. Additionally, such synthetic cationic deposition polymers have a charge density from about 0.1 meq/g to about 5.0 meq/g.

Suitable synthetic cationic deposition polymers include those which are water-soluble or dispersible, cationic, non-crosslinked, conditioning copolymers comprising: (i) one or more cationic monomer units; and (ii) one or more nonionic monomer units or monomer units bearing a terminal negative charge; wherein said copolymer has a net positive charge, a cationic charge density of from about 0.5 meq/g to about 10 meg/g, and an average molecular weight from about 1,000 to about 5,000,000.

Non-limiting examples of suitable synthetic cationic deposition polymers are described in United States Patent Application Publication US 2003/0223951 A1 to Geary et al.

Anti-Dandruff Actives

The compositions of the present invention may also contain an anti-dandruff active. Suitable non-limiting examples of anti-dandruff actives include pyridinethione salts, azoles, selenium sulfide, particulate sulfur, keratolytic agents, and mixtures thereof. Such anti-dandruff actives should be physically and chemically compatible with the essential components of the composition, and should not otherwise unduly impair product stability, aesthetics or performance.

Pyridinethione anti-microbial and anti-dandruff agents are described, for example, in U.S. Pat. Nos. 2,809,971; 3,236,733; 3,753,196; 3,761,418; 4,345,080; 4,323,683; 4,379,753; and 4,470,982.

Azole anti-microbials include imidazoles such as climbazole and ketoconazole.

Selenium sulfide compounds are described, for example, in U.S. Pat. Nos. 2,694,668; 3,152,046; 4,089,945; and 4,885,107.

Sulfur may also be used as a particulate anti-microbial/anti-dandruff agent in the anti-microbial compositions of the present invention.

The present invention may further comprise one or more keratolytic agents such as salicylic acid. In a preferred embodiment, salicylic acid provides chemical exfoliation activity.

Additional anti-microbial actives of the present invention may include extracts of melaleuca (tea tree) and charcoal.

When present in the composition, the anti-dandruff active is included in an amount from about 0.01% to about 5%, preferably from about 0.1% to about 3%, and more preferably from about 0.3% to about 2%, by weight of the composition.

Particles

The compositions of the present invention optionally may comprise particles. Preferably, particles useful in the present invention are dispersed water-insoluble particles. Particles useful in the present invention can be inorganic, synthetic, or semi-synthetic. In the compositions of the present invention, it is preferable to incorporate no more than about 20%, more preferably no more than about 10% and even more preferably no more than 2%, by weight of the composition, of particles. In an embodiment of the present invention, the particles have an average mean particle size of less than about 300 μm.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles include silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

Opacifying Agents

The compositions of the present invention may also contain one or more opacifying agents. Opacifying agents are typically used in cleansing compositions to impart desired aesthetic benefits to the composition, such as color or pearlescence. In the compositions of the present invention, it is preferable to incorporate no more than about 20%, more preferably no more than about 10% and even more preferably no more than 2%, by weight of the composition, of opacifying agents.

Suitable opacifying agents include, for example, fumed silica, polymethylmethacrylate, micronized Teflon®, boron nitride, barium sulfate, acrylate polymers, aluminum silicate, aluminum starch octenylsuccinate, calcium silicate, cellulose, chalk, corn starch, diatomaceous earth, Fuller's earth, glyceryl starch, hydrated silica, magnesium carbonate, magnesium hydroxide, magnesium oxide, magnesium trisilicate, maltodextrin, microcrystaline cellulose, rice starch, silica, titanium dioxide, zinc laurate, zinc myristate, zinc neodecanoate, zinc rosinate, zinc stearate, polyethylene, alumina, attapulgite, calcium carbonate, calcium silicate, dextran, nylon, silica silylate, silk powder, soy flour, tin oxide, titanium hydroxide, trimagnesium phosphate, walnut shell powder, or mixtures thereof. The above-mentioned powders may be surface treated with lecithin, amino acids, mineral oil, or various other agents either alone or in combination, which coat the powder surface and render the particles hydrophobic in nature.

The opacifying agents may also comprise various organic and inorganic pigments. The organic pigments are generally various aromatic types including azo, indigoid, triphenylmethane, anthraquinone, and xanthine dyes. Inorganic pigments include iron oxides, ultramarine and chromium or chromium hydroxide colors, and mixtures thereof.

Suspending Agents

The compositions of the present invention may further comprise a suspending agent at concentrations effective for suspending water-insoluble material in dispersed form in the compositions or for modifying the viscosity of the composition. Such concentrations generally range from about 0.1% to about 10%, preferably from about 0.3% to about 5.0%, by weight of the composition, of suspending agent.

Suspending agents useful herein include anionic polymers and nonionic polymers. Useful herein are vinyl polymers such as cross-linked acrylic acid polymers with the CTFA name Carbomer.

Paraffinic Hydrocarbons

The compositions of the present invention may contain one or more paraffinic hydrocarbons. Paraffinic hydrocarbons suitable for use in compositions of the present invention include those materials which are known for use in hair care or other personal care compositions, such as those having a vapor pressure at 1 atm of equal to or greater than about 21° C. (about 70° F.). Non-limiting examples include pentane and isopentane.

Propellants

The composition of the present invention also may contain one or more propellants. Propellants suitable for use in compositions of the present invention include those materials which are known for use in hair care or other cosmetic compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

Other Optional Components

The compositions of the present invention may contain fragrance.

The compositions of the present invention may also contain water-soluble and water-insoluble vitamins such as vitamins B1, B2, B6, B12, C, pantothenic acid, pantothenyl ethyl ether, panthenol, biotin and their derivatives, and vitamins A, D, E, and their derivatives. The compositions of the present invention may also contain water-soluble and water-insoluble amino acids such as asparagine, alanine, indole, glutamic acid and their salts, and tyrosine, tryptamine, lysine, histadine and their salts.

The compositions of the present invention may contain a mono- or divalent salt such as sodium chloride.

The compositions of the present invention may also contain chelating agents.

The compositions of present invention may further comprise materials useful for hair loss prevention and hair growth stimulants or agents.

Conditioner

The composition may comprise one or more conditioning agents. Preferably, the actives are natural or naturally derived actives selected from starches, guars, non-guar galactomannan polymer derivatives, plant extracts, and the like.

Starches suitable for the compositions are those which generally result from any vegetable source. Nonlimiting examples include corn, potato, the oats, rice, tapioca, the sorghum, the barley or corn.

The conditioning actives are used preferably in an amount of from 0.01 to 20% in weight compared to the total weight of the composition. More preferably, from 0.05 to 15% in weight compared to the total weight of the composition and even more preferably from 0.1 to 10% by weight of the composition.

The hair conditioning compositions may also comprise non-guar galactomannan polymer derivatives having a mannose to galactose ratio of greater than 2:1 on a monomer to monomer basis, the non-guar galactomannan polymer derivative is selected from the group consisting of a cationic non-guar galactomannan polymer derivative and an amphoteric non-guar galactomannan polymer derivative having a net positive charge. As used herein, the term “cationic non-guar galactomannan” refers to a non-guar galactomannan polymer to which a cationic group is added. The term “amphoteric non-guar galactomannan” refers to a non-guar galactomannan polymer to which a cationic group and an anionic group are added such that the polymer has a net positive charge. Non-guar galactomannan polymer derivatives provide improved efficacy of conditioning agents. Further, the non-guar galactomannan polymer derivatives have been found to reduce overall viscosity of conditioning compositions, which results in improved feel benefits.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or carob (4 parts mannose/1 part galactose), and cassia gum (5 parts mannose/1 part galactose). A preferred non-guar galactomannan polymer derivative is cationic cassia.

The cationic non-guar galactomannan polymer derivatives have a molecular weight from about 1,000 to about 10,000,000. In one embodiment of the present invention, the cationic non-guar galactomannan polymer derivatives have a molecular weight from about 5,000 to about 3,000,000. As used herein, the term “molecular weight” refers to the weight average molecular weight. The weight average molecular weight may be measured by gel permeation chromatography.

The hair conditioning compositions of the present invention may include non-guar galactomannan polymer derivatives which have a cationic charge density from about 0.7 meq/g to about 7 meq/g. In one embodiment of the present invention, the non-guar galactomannan polymer derivatives have a charge density from about 0.9 meq/g to about 7 meq/g. The degree of substitution of the cationic groups onto the non-guar galactomannan structure should be sufficient to provide the requisite cationic charge density.

In one embodiment of the present invention, the non-guar galactomannan polymer derivative is a cationic derivative of the non-guar galactomannan polymer, which is obtained by reaction between the hydroxyl groups of the non-guar galactomannan polymer and reactive quaternary ammonium compounds

In another embodiment of the present invention, the non-guar galactomannan polymer derivative is an amphoteric non-guar galactomannan polymer derivative having a net positive charge, obtained when the cationic non-guar galactomannan polymer derivative further comprises an anionic group.

The hair conditioning compositions may comprise non-guar galactomannan polymer derivatives at a range of from about 0.01% to about 10%, and more preferably from about 0.05% to about 5%, by weight of the composition.

The compositions may further include one or more conditioning polymers selected from derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the composition of this invention at a concentration of from 0.1 to 10 weight percent, preferably from 0.2 to 6 weight percent and most preferably from 0.2 to 5 weight percent.

Conditioning Agents The conditioning compositions may also comprise one or more conditioning agents, such as those selected from the group consisting of cationic surfactants, cationic polymers, nonvolatile hydrocarbons, saturated C14 to C22 straight chain fatty alcohols, nonvolatile hydrocarbon esters, and mixtures thereof. Preferred conditioning agents are cationic surfactants, cationic polymers, saturated C14 to C22 straight chain fatty alcohols, and quarternary ammonium. Plant extracts such as ginseng root extract, silybaum marianum extract, phyllanthus emblica fruit extract, and the like are also suitable. The components hereof can comprise from about 0.1% to about 99%, more preferably from about 0.5% to about 90%, of conditioning agents. However, in the presence of an aqueous carrier, the conditioning agents preferably comprise from about 0.1% to about 90%, more preferably from about 0.5 to about 60% and most preferably from about 1% to about 50% by weight of the hair conditioning composition.

The conditioning compositions also include one or more natural stimulants in order to stimulate the scalp prior to application of the serum component. Exemplary natural stimulants include those such as ginseng and caffeine.

Cationic Surfactants

Cationic surfactants, useful in the compositions, contain amino or quaternary ammonium moieties. The cationic surfactant will preferably, though not necessarily, be insoluble in the compositions hereof. Cationic surfactants among those useful herein are disclosed in the following documents: M.C. Publishing Co., McCutcheoris, Detergents Sc Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983. Among the quaternary ammonium-containing cationic surfactant materials useful herein are those of the general formula:

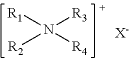

wherein R1-R4 are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate nitrate, sulfate, and alkylsulfate radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated. Especially preferred are di-long chain (e.g., di C12-22, preferably C14-C20, aliphatic, preferably alkyl) di-short chain (e.g., C1-C3 alkyl, preferably C1-C2 alkyl) and quaternary ammonium salts. Salts of primary, secondary and tertiary fatty amines are also suitable cationic surfactant materials. The alkyl groups of such amines preferably have from about 12 to about 22 carbon atoms and may be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981. Cationic surfactants are preferably utilized at levels of from about 0.1% to about 10%, more preferably from about 0.25% to about 5%, most preferably from about 0.5% to about 2%, by weight of the composition.

### Non-Limiting Examples

The compositions illustrated in the following Examples illustrate specific embodiments of the compositions of the present invention but are not intended to be limiting thereof. Other modifications can be undertaken by the skilled artisan without departing from the spirit and scope of this invention.

The compositions according to the following Examples may be prepared according to the following steps:

A=oil phase; combine and heat to 80 C.
B=water phase; combine and heat to 80 C. when both A and B reach 80 C, combine and mix until homogenous.
C=add after batch has been cooled below 35 C. homogenize batch for optimum particle size and viscosity.

All exemplified amounts are listed as weight percents and exclude minor materials such as diluents, preservatives, color solutions, imagery ingredients, botanicals, and so forth, unless otherwise specified.

The following Examples are representative of cosmetic compositions of the present invention:

TABLE 1

| Phase | Ingredient | Ex 1 | Ex 2 | Ex 3 | Ex 4 | Ex 5 | Ex 6 |
|---|---|---|---|---|---|---|---|
| A | CETEARYL | 7.00 | 7.00 | 7.00 | 7.00 | 7.00 | 7.00 |
| | ALCOHOL/BEHENTRIMONIUM | | | | | | |
| | METHOSULFATE | | | | | | |
| A | CETEARYL ALCOHOL | 2.80 | 2.80 | 2.80 | 2.80 | 2.80 | 2.80 |
| A | CAPRYLYL GLYCOL | 0.30 | 0.30 | 0.30 | 0.30 | 0.30 | 0.30 |
| A | TRICAPRYLYL CITRATE | 1.75 | 0.5 | 0.10 | 1.25 | 1.50 | 1.00 |
| A | HEPTYL UNDECYLENATE | 1.75 | 2.00 | 0.50 | 0.10 | 1.00 | 1.50 |
| A | POLYGLYCERYL-10 | 1.00 | 2.00 | 1.50 | 0.50 | 0.50 | 0.20 |
| | PENTAOLEATE | | | | | | |
| A | OLIVE | 2.00 | 1.00 | 1.50 | 0.75 | 0.05 | 1.75 |
| | SQUALANE | | | | | | |
| A | CANDELILLA WAX | 1.75 | 0.50 | — | 1.75 | 1.50 | 1.00 |
| A | CARNAUBA WAX | 0.50 | 1.75 | 0.50 | — | — | 1.00 |
| A | NATURAL BEESWAX | 0.50 | — | 1.75 | 0.5 | 1.00 | — |
| A | SHEA BUTTER | 1.00 | 1.00 | 1.00 | 1.00 | 1.00 | 1.00 |
| A | COCONUT OIL | 1.00 | 1.00 | 1.00 | 1.00 | 1.00 | 1.00 |
| B | POLYQUATERNIUM-10 | 0.15 | 0.15 | 0.15 | 0.15 | 0.15 | 0.15 |
| B | CETRIMONIUM CHLORIDE | 2.50 | 2.50 | 2.50 | 2.50 | 2.50 | 2.50 |
| B | DICETYLDIMONIUM | 2.00 | 2.00 | 2.00 | 2.00 | 2.00 | 2.00 |
| | CHLORIDE | | | | | | |
| B | POTASSIUM SORBATE | 0.10 | 0.10 | 0.10 | 0.10 | 0.10 | 0.10 |
| B | LACTIC ACID | 0.82 | 0.82 | 0.82 | 0.82 | 0.82 | 0.82 |
| B | STEARAMIDOPROPYL | 2.00 | 2.00 | 2.00 | 2.00 | 2.00 | 2.00 |
| | DIMETHYLAMINE | | | | | | |
| B | PROPANEDIOL | 1.00 | 1.00 | 1.00 | 1.00 | 1.00 | 1.00 |
| B | WATER | qs to | qs to | qs to | qs to | qs to | qs to |
| | | 100 | 100 | 100 | 100 | 100 | 100 |
| C | PHENOXETOL | 0.30 | 0.30 | 0.30 | 0.30 | 0.30 | 0.30 |
| | PHENOXYETHANOL | | | | | | |
| C | BENZYL ALCOHOL CP/NF | 0.95 | 0.95 | 0.95 | 0.95 | 0.95 | 0.95 |
| | BENZYL ALCOHOL | | | | | | |

| | | | Perferred Usage |
|---|---|---|---|
| INCI NAME | Min % | Max % | Range (%) |
| Tricaprylyl Citrate¹ | 0.01 | 20 | 0.10-1.75 |
| Heptyl Undecylenate² | 0.01 | 20 | 0.10-1.75 |
| Squalane*³ | 0.01 | 20 | 0.05-2.00 |
| Euphorbia Cerifera (Candelilla) | 0.01 | 20 | 0.05-1.75 |
| Wax⁴ | | | |

¹Available under the Tradename Bernel Ester TCC
²Available under the Tradename Lexfeel Natural
³Available under the Tradename Olive Squalane*
⁴Available under the Tradename Candelilla Wax Flakes

| | | | | Preferred |
|---|---|---|---|---|
| | | | | Usage Range |
| INCI Name | Trade Name | Min % | Max % | (%) |
| Polyglyceryl-10 | Barglide 50* | 0.01 | 20 | 0.20-2.00 |
| Pentaoleate* | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for forming a hair conditioning composition comprising the steps of:
a. forming a first phase, said first phase comprising an olive squalane, one or more esters, and one or more waxes;
b. forming a second phase, said second phase comprising one or more non-silicone conditioning agents, one or more cationic polymers, and water;
c. combining said first and second phase to form a mixture;
d. adding one or more preservatives to said mixture; and.
wherein said composition is free of silicone oils.

2. The method for forming a hair conditioning composition according to claim 1, wherein said composition is substantially free of one or more components selected from the group consisting of anionic surfactants, amidoamines, silicone derivatives, ethoxylated emulsifiers, and mixtures thereof.

3. The method for forming a hair conditioning composition according to claim 1, further comprising from about 0.05 wt. % to about 5 wt. % of a cellulose or glactomannan cationic deposition polymer having a molecular weight greater than about 200,000 and a charge density from about 0.15 meq/g to about 4.0 meq/g.

4. The method for forming a hair conditioning composition according to claim 1, wherein said olive squalane is present at from about 0.05 wt. % to about 2 wt. % of said composition.

5. The method for forming a hair conditioning composition according to claim 1, wherein said wax is selected from the group consisting of microcrystalline wax, paraffin wax, microcrystalline wax, ozokerite, polyethylene wax, ceresin wax, bees wax, candelilla wax, carnauba wax, shellac wax, lanolin wax, montan wax, orange wax, lemon wax, laurel wax and olive wax, beeswax, and mixtures thereof.

6. The method for forming a hair conditioning composition according to claim 5, wherein said wax is present at from about 0.05 wt. % to about 2 wt. % of said composition.

7. The method for forming a hair conditioning composition according to claim 1, wherein said ester is selected from the group consisting of methylheptyl isostearate, diisostearyl dimer dilinoleate, diisostearyl malate, tricaprylyl citrate, isostearyl isostearate, polyglyceryl-2 triisostearate, polyglyceryl-2 diisostearate, polyglyceryl-2 isostearate, glyceryl ricinoleate, isostearyl palmitate, caprylic/capric triglyceride, mango butter dimer dilinoleyl esters/dimer dilinoleate copolymer, myristyl myristate, oleyl erucate, polyglyceryl-3 diisostearate, triisostearyl citrate, isostearyl stearoyl stearate, Mangifera indica (mango) seed butter, tricaprylin, glucose glutamate, and mixtures thereof.

8. The method for forming a hair conditioning composition according to claim 7, wherein said ester is tricaprylyl citrate.

9. The method for forming a hair conditioning composition according to claim 8, wherein said tricaprylyl citrate is present at from about 0.05 wt. % to about 2 wt. % of said composition.

10. The method for forming a hair conditioning composition according to claim 1, further comprising one or more additional components selected from the group consisting of dispersed water-insoluble particles, opacifying agents, suspending agents, anti-dandruff agents, non-volatile paraffinic hydrocarbons, propellants, and mixtures thereof.
